(19) United States
(12) Patent Application Publication
(10) Pub. No.: US 2007/0065522 A1
(43) Pub. Date: Mar. 22, 2007

(54) Administration of high potency platinum compound formulations by inhalation

(75) Inventors: Frank G. Pilkiewicz, Princeton Junction, NJ (US); Walter Perkins, Pennington, NJ (US); Beth Metzheiser, Cranbury, NJ (US)

Correspondence Address: FOLEY HOAG, LLP;PATENT GROUP, WORLD TRADE CENTER WEST, 155 SEAPORT BLVD, BOSTON, MA, 02110, US

(73) Assignee: Transave, Inc., Monmouth Junction, NJ (US)

(21) Appl. No.: 11/505,236

(22) Filed: Aug. 16, 2006

Related U.S. Application Data
(63) Continuation-in-part of application No. 11/084,070, filed on Mar. 18, 2005.
(60) Provisional application No. 60/554,262, filed on Mar. 18, 2004.
(60) Provisional application No. 60/573,521, filed on May 21, 2004.

(51) Int. Cl. A61K 33/24; A61K 31/555; A61K 31/282

(52) U.S. Cl. 424/649; 514/492; 514/184

(57) ABSTRACT

Provided is a method for treating a patient having cancer. The method includes administering high potency lipid-platinum compound formulations to the patient's respiratory tract. Also provided is a method of reducing treatment times for administering platinum compound formulations to a patient in need thereof by administering high potency lipid-platinum compound formulations to the patient.

### RELATED APPLICATIONS

This application is a continuation-in-part of and claims the benefit of priority to U.S. application Ser. No. 11/084,070, filed Mar. 18, 2005, which claims priority to U.S. Provisional Application Ser. No. 60/554,262, filed Mar. 18, 2004, and U.S. Provisional Application Ser. No. 60/573,521, filed May 21, 2004; the entirety of which are hereby incorporated by reference.

### BACKGROUND OF THE INVENTION

The present invention relates to a method for treating cancer by delivering a therapeutically effective amount of a lipid composition containing a cytotoxic agent (e.g., cisplatin) to a patient's respiratory tract. The method allows clinicians to administer treatment cycles more frequently without the attendant side effects (e.g., nephrotoxicity, bone marrow toxicity) common to systemic administration of many cancer cytotoxic agents (e.g., cisplatin).

Bronchoalveolar Carcinoma (BAC) or alveolar cell carcinoma is a form of adenocarcinoma, a cell-type of non-small cell carcinoma of the lung which can be found throughout the respiratory tract. BAC represents approximately 10 to 25% of the adenocarcinoma of lung cases or 2-6% of all lung cancers and sometimes has a distinct presentation and biologic behavior. BAC is more common in women and in patients who do not smoke cigarettes than other histologic types of lung cancer.

BAC may present as a solitary peripheral nodule, a multifocal lesion, or a rapidly progressive form that appears as a diffuse infiltrate on chest radiograph. The cells secrete mucin and surfactant apoprotein which can lead to bronchorrhea, an excessive discharge of mucus from the air passages of the lungs. Bronchoalveolar cancer may present as a more diffuse lesion than other types of cancer. When it is discovered as a single mass on a patient's x-ray, this type of lung cancer has an excellent prognosis. Five year survival after surgery is in the 75-90 percent range. If, however, it is found in its diffuse form (meaning it has spread beyond a single mass), the prognosis is quite poor.

The management and prognosis are essentially the same as other types of non-small cell lung cancer. Surgery is the preferred treatment if the tumor can be resected. Radiation therapy and chemotherapy may be used in non-operable cases. Trials are underway to investigate treatments specific for bronchoalveolar carcinoma.

Carcinomatosis with lymphangitic spread, or Lymphangitis carcinomatosis (LC) refers to the diffuse infiltration and obstruction of pulmonary parenchymal lymphatic channels by tumor. Various neoplasms can cause lymphangitic carcinomatosis, but 80% are adenocarcinomas. The most frequent primary sites are the breast, lungs, colon, and stomach. Other sources include the pancreas, thyroid, cervix, prostate, larynx, and metastatic adenocarcinoma from an unknown primary.

LC occurs as a result of initial hematogenous spread of tumor to the lungs, with subsequent malignant invasion through the vessel wall into the pulmonary interstitium and lymphatics. The tumor then proliferates and spreads easily through these low resistance channels. Less commonly, direct infiltration occurs from contiguous mediastinal or hilar lymphadenopathy or from an adjacent primary bronchogenic carcinoma. Histopathologically, interstitial edema, interstitial fibrosis (secondary to a desmoplastic reaction as a result of tumor extension into adjacent pulmonary parenchyma), and tumor cells all can be seen. Metastatic adenocarcinoma accounts for 80% of cases. Most patients are middle-aged adults.

In the United States, LC represents 7% of all pulmonary metastases. Prevalence in postmortem studies is significantly higher than the incidence of radiologically detectable disease. Microscopic interstitial tumor invasion is seen in 56% of patients with pulmonary metastases. Prognosis for patients with LC is poor. Most patients survive only weeks or months.

Osteosarcoma commonly metastasizes to the lung. The presence of lung metastases has a major impact on the prognosis of patients with osteosarcoma. Upon surgical removal of the tumor in the lung, new pulmonary metastases often recur within months suggesting micro-metastatic disease resistant to systemic chemotherapy. Liposomal cisplatin offers the potential ability to attain a prolonged therapeutic effect of cisplatin in the lung by sustained release. The ability to give liposomal cisplatin by inhalation directly to the lung permits high drug levels at the site of disease with low systemic exposure.

Typically, chemotherapeutic treatment of lung cancers includes systemic administration of chemotherapeutic agents, e.g., cytotoxic agents, to the patients. Often such administration, e.g., intravenous administration, is associated with several adverse side effects including nephrotoxicity and bone marrow toxicity. For instance, systemic administration of cisplatin (cis-diamine-dichloroplatinum (II)) one of the more effective anti-tumor agents used in the systemic treatment of lung cancers, is often burdened by symptoms such as nephrotoxicity in the patient. The nephrotoxicity limits the frequency in which clinicians can administer cisplatin to the patient. In fact, successive treatment cycles of cisplatin typically require three weeks or more between treatment cycles to prevent blood levels of cisplatin from reaching those correlated with nephrotoxicity. Since chemotherapeutic regimens typically require five or more treatment cycles, the delay between treatment cycles lengthens the time needed for the overall chemotherapeutic regimen. The prolonged time periods for systemic administration of cisplatin lead to increased patient discomfort and inconvenience, and may lead to decreased patient compliance.

Attempts to minimize the toxicity of active platinum compounds have included combination chemotherapy, synthesis of analogues (Prestayko et al., 1979 Cancer Treat Rev. 6(1): 17-39; Weiss, et al., 1993 Drugs. 46(3): 360-377), immunotherapy and entrapment in liposomes (Sur, et al., 1983; Weiss, et al., 1993). Antineoplastic agents, including cisplatin, entrapped in liposomes have a reduced toxicity, relative to the agent in free form, while retaining antitumor activity (Steerenberg, et al., 1987; Weiss, et al., 1993).

Cisplatin, however, is difficult to efficiently entrap in liposomes or lipid complexes because of the bioactive agent's low aqueous solubility, approximately 1.0 mg/ml at room temperature, and low lipophilicity, both of which properties contribute to a low bioactive agent/lipid ratio.

Liposomes and lipid complexes containing cisplatin suffer from another problem—stability of the composition. In particular, maintenance of bioactive agent potency and retention of the bioactive agent in the liposome during storage are recognized problems (Freise, et al., 1982; Gondal, et al., 1993; Potkul, et al., 1991 Am J Obstet Gynecol. 164(2): 652-658; Steerenberg, et al., 1988; Weiss, et al., 1993) and a limited shelf life of liposomes containing cisplatin, on the order of several weeks at 4° C., has been reported (Gondal, et al., 1993 Eur J Cancer. 29A(11): 1536-1542; Potkul, et al., 1991).

Accordingly, new methods for treating patients suffering from lung cancer by inhalation administration of cisplatin that allow significant local concentrations of drug to be attained by shortening of the time periods needed between treatment cycles are desirable. Such methods preferably also overcome the rapid clearance of cisplatin from the lung that typically plague inhalation administration of therapeutic agents.

### SUMMARY OF THE INVENTION

It is an object of the present invention to provide a method of treating a patient for cancer by inhalation of platinum compound formulations with decreased administration times for greater patient comfort and compliance.

It is also an object of the present invention to provide a method of reducing treatment times for a patient with cancer undergoing treatments with platinum compound formulations by inhalation.

The present invention results from the realization that effective treatment with reduced administration times can be achieved with high potency platinum compound formulations. By high potency, it is meant that the concentration of the platinum compound is higher than its aqueous solubility. For example, for cisplatin, high potency would be concentrations higher than its aqueous solubility of 1 mg/ml. Platinol®, for example, is a commercial cisplatin product by Bristol Meyers Squibb having 1 mg/ml cisplatin.

In one embodiment, the present invention relates to a method of treating cancer or a method of reducing treatment times for a patient comprising administering to the patient by inhalation, a cancer treating effective amount of a lipid-based platinum compound formulation wherein the concentration of the platinum compound of the lipid-based platinum compound formulation is greater than its aqueous solubility.

In a further embodiment, the platinum compound concentration is 1.2 mg/ml, 3 mg/ml, or 5 mg/ml.

The platinum compound is selected from the group consisting of: cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, DACH-platinum such as oxaliplatin and aroplatin, heptaplatin, iproplatin, transplatin, lobaplatin, cis-aminedichloro(2-methylpyridine) platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof. In a preferred embodiment, the platinum compound is cisplatin.

The lipids used in the present invention can be synthetic, semi-synthetic or naturally-occurring lipids, including phospholipids, tocopherols, sterols, fatty acids, glycolipids, anionic lipids, cationic lipids. In terms of phosholipids, they can include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), sterically modified phosphatidylethanolamines, cholesterol derivatives, carotinoids, other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC, a major constituent of naturally-occurring lung surfactant. Other examples include dimyristoylphosphatidycholine (DMPC) and dimyristoylphosphatidylg-lycerol (DMPG) dipalmitoylphosphatidcholine (DPPC and dipalmitoylphosphatidylglycerol (DPPG) distearoylphosphatidylcholine (DSPC and distearoylphosphatidylglycerol (DSPG), dioleylphosphatidyl-ethanolamine (DOPE) and mixed phospholipids like palmitoylstearoylphosphatidylcholine (PSPC) and palmitoylstearolphosphatidylglycerol (PSPG), triacylglycerol, diacylglycerol, sphingosine, ceramide, sphingomyelin, single acylated phospholipids like mono-oleoyl-phosphatidylethanolarnine (MOPE), and quadruple acylated.phospholipids like cardiolipin.

In a preferred embodiment, the lipid in the lipid-based platinum compound formulation is a mixture of a phospholipid and a sterol, such as for example, DPPC and cholesterol. In a preferred embodiment, the lipid in the lipid-based platinum compound formulation is a mixture of DPPC from 50 to 65 mol % and cholesterol from 35 to 50 mol %.

The cancer, in one embodiment, may be lung cancer, such as, for example, BAC or LC.

In a preferred embodiment, the ratio of platinum compound to lipid in the lipid-based platinum compound formulation is between 1:5 by weight and 1:50 by weight. In still a another embodiment, the lipid-based platinum compound formulation comprises liposomes having a mean diameter of 0.01 microns to 3.0 microns.

In a preferred embodiment, the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns.

In a preferred embodiment, the lipid is a mixture of DPPC and cholesterol, the ratio of platinum compound to lipid in the lipid-based platinum compound formulation is between 1:5 by weight and 1:50 by weight, the lipid-based platinum compound formulation comprises liposomes having a mean diameter of 0.01 microns to 3.0 microns, and wherein the platinum compound is cisplatin.

In a preferred embodiment, the lipid is a mixture of DPPC and cholesterol in a 2 to 1 ratio by weight, the ratio of platinum compound to lipid in the lipid-based platinum compound formulation is 1:20 by weight, the lipid-based platinum compound formulation comprises liposomes having a mean diameter of 0.40 microns, and wherein the platinum compound is cisplatin.

The patient is preferably a human.

In a preferred embodiment, the lipid-based platinum compound formulation is administered to the patient at least once every three weeks.

In a preferred embodiment, the amount of platinum compound in the lipid-based platinum compound formulation is 18 mg/m² or greater, 24 mg/m² or greater, 36 mg/m² or greater, or 48 mg/m² or greater.

In a preferred embodiment, the amount of platinum compound in the lipid-based platinum compound formulation is 100 mg/m² or greater, and the lipid-based platinum compound formulation is administered to the patient at least once every three weeks, and preferably once every two weeks.

These embodiments of the present invention, other embodiments, and their features and characteristics, will be apparent from the description and claims that follow.

### BRIEF DESCRIPTION OF THE DRAWINGS

FIG. 1 depicts the aerodynamic particle size distributions of liposomal cisplatin lots at different potencies: 1 mg/ml and 5 mg/ml.

FIG. 2 depicts Pt levels in the lungs of rats as a function of time after inhalation. Whole lungs were homogenized. Points represent the mean of three samples±s.d.

FIG. 3 depicts Pt levels in the lymph nodes of rats as a function of time after inhalation. Whole bronchial lymph nodes were homogenized. Points represent the mean of three samples±s.d.

### DETAILED DESCRIPTION OF THE INVENTION

Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

A “platinum compound” is a compound containing coordinated platinum and having antineoplastic activity. Active platinum compounds include, for example, cisplatin, carboplatin, and DACH-platinum compounds such as oxaplatin and aroplatin.

A “patient,” “subject” or “host” to be treated by the subject method may mean either a human or non-human animal.

The term “therapeutic effect” is art-recognized and refers to a local or systemic effect in animals, particularly mammals, and more particularly humans caused by a pharmacologically active substance. The phrase “therapeutically-effective amount” means that amount of a substance that produces some desired local or systemic effect at a reasonable benefit/risk ratio applicable to any treatment. The therapeutically effective amount of a substance will vary depending upon the subject and disease condition being treated, the weight and age of the subject, the severity of the disease condition, the manner of administration and the like, which can readily be determined by one of ordinary skill in the art.

The term “treating” is art-recognized and refers to curing as well as ameliorating at least one symptom of a condition or disease or preventing the occurrence of a condition or disease.

“Treatment cycle” means the time period in which a given dose of cisplatin is to be administered to a patient. Treatment cycles may encompass one or more sessions where the patient is actively being administered the lipid composition containing cisplatin. Such sessions may be administered over the course of four days or less, but more preferably is administered over one or two days, and most preferably is administered in one day.

High Potency Platinum Compound Formulations

High potency lipid formulations of platinum compounds have been designed for aerosol administration to patients with primary and metastatic cancer in the lungs. Aerosolized platinum compound formulations enable effective targeting of the drug to the lungs without significant systemic levels. The improved lung targeting reduces systemic toxicity and enables larger doses of therapeutic to be delivered.

One consequence of the ability to deliver high doses of chemotherapeutics via inhalation is the potential for extended administration times, which can potentially impact patient compliance and make the procedure less attractive for caregivers to administer. Currently, it takes approximately 160 min to administer a dose of 24 mg/m² (assuming a 2 m² patient), with Lot A (1 mg/ml cisplatin), PARI LC Star nebulizer, and Devilbiss 8650D compressor. This is done in three sessions spread throughout a day. This intense therapy can be taxing, and some patients have difficulty in completing the procedure in a single day. Hence, it would be advantageous to be able to diminish the nebulization times.

A critical success factor for administering a clinically therapeutic dose of a platinum compound formulation would be to do so in no more than 1 hr. Preferably, this would be accomplished without a formulation or device change. One potential way to do this is to increase the potency of the drug product (i.e., reduce water content from the product). In the present invention, the aerodynamic particle size distribution of a more concentrated formulation of cisplatin, Lot B (5 mg/ml cisplatin), in comparison with Lot A (1 mg/ml cisplatin) has been examined. Provided the aerosol characteristics of the two products are equivalent, the five-fold more potency would enable doses up to 24 mg/m² to be delivered in less than the 1 hr target (ca., 32 min).

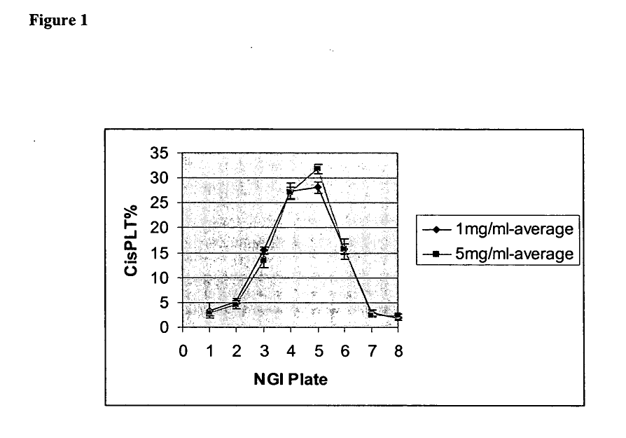

The aerodynamic particle size distributions for 1 mg/ml and 5 mg/ml cisplatin formulations are depicted in FIG. 1. The values shown represent the means of three independent runs and the error bars represent the standard deviations (S.D.). The pattern of the particle size distributions is virtually indistinguishable for the two formulations.

The mass median aerodynamic diameters (MMAD) and geometric standard deviations (GSD) for the formulations are presented in Table 1. The MMAD values range from about 3.3 μm to 3.9 μm. The mean MMAD values for the 1 mg/ml and 5 mg/ml formulations are 3.7 and 3.6 μm, respectively (n=3). The mean GSD values for the 1 mg/ml and 5 mg/ml formulations are 1.94 and 1.89, respectively.

TABLE 1
The measured MMAD and GSD values for 1 mg/ml and
5 mg/ml formulations of liposomal cisplatin.

| | |
|---|---|
| Lot A (1 mg/ml) | Lot B (5 mg/ml) |

| | MMAD (μm) | GSD | MMAD (μm) | GSD |
|---|---|---|---|---|
| Run 1 | 3.86 | 1.96 | 3.33 | 1.89 |
| Run 2 | 3.63 | 1.91 | 3.72 | 1.9 |
| Run 3 | 3.64 | 1.95 | 3.64 | 1.88 |
| Mean | 3.71 | 1.94 | 3.56 | 1.89 |
| S.D. | 0.13 | 0.03 | 0.21 | 0.01 |

The rates of nebulization for the formulations are presented in Table 2. The rate of nebulization range from about 0.26 g/min to 0.39 g/min. The mean rate of nebulization for the 1 mg/ml and 5 mg/ml formulations are 0.36 g/min and 0.34 g/min, respectively (n=3). This indicates that there is no significant difference in rate of nebulization between the two formulations.

TABLE 2
The measured rate of nebulization for 1 mg/ml and
5 mg/ml formulations of liposomal cisplatin.

| | Lot A (1 mg/ml) | Lot B (5 mg/ml) |
|---|---|---|
| | Rate of Nebulization (g/min) | Rate of Nebulization (g/min) |
| Run 1 | 0.3 | 0.26 |
| Run 2 | 0.39 | 0.38 |
| Run 3 | 0.38 | 0.38 |
| Mean | 0.36 | 0.34 |
| S.D. | 0.05 | 0.07 |

Increasing the potency of liposomal cisplatin from 1 mg/ml to 5 mg/ml does not result in significant differences in the aerodynamic particle size distribution of the nebulized droplets. Increasing the potency of liposomal cisplatin from 1 mg/ml to 5 mg/ml does not result in significant differences in the rate of nebulization. Hence, it is anticipated that the increase in potency will enable five-fold reductions in administration time.

Kinetics and Bioavailability

The kinetics and bioavailability of liposomal cisplatin (Lot A: 1.0 mg/ml cisplatin) versus that of liposomal cisplatin (Lot C: 1.7 mg/ml cisplatin) when administered by inhalation were carried out and compared to inhaled soluble cisplatin.

The delivery of Pt to the lymph nodes of animals receiving the drug was determined as well as that to the lung because the bronchial lymph nodes are a primary site of metastasis of lung tumors. Delivery of cisplatin to the lymph nodes may be a means to reduce metastatic spread.

Rats received either nebulized 1.7 mg/ml liposomal cisplatin (Lot C) via inhalation for 60 minutes or nebulized 1.0 mg/ml liposomal cisplatin (Lot A) via inhalation for 90 minutes or nebulized 1.0 mg/ml free cisplatin via inhalation for 90 minutes. Tissue samples were taken at various times after the end of inhalation.

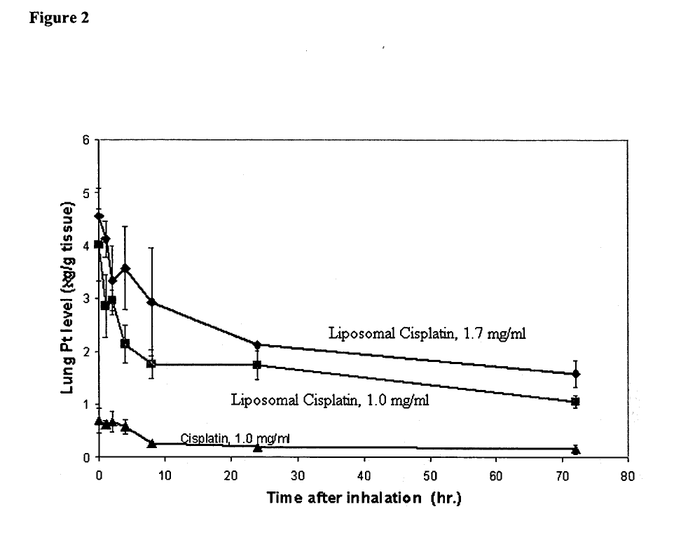

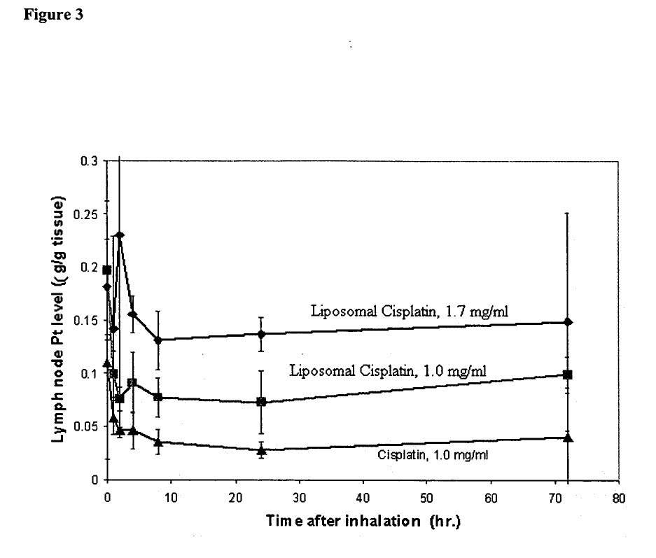

Time courses of Pt levels in various tissues are shown in FIGS. 2 and 3. The kinetics of clearance of Pt from either the high or low liposomal cisplatin concentrations is similar in the lung (FIG. 2). A slightly higher Cₘₐₓ and AUC may reflect the slightly higher dose under these conditions.

These results suggest that the two formulations of liposomal cisplatin have nearly equal bioavailability and pharmacokinetics when administrated by inhalation. A marked sustained release of Pt in the lung is observed for liposomal cisplatin formulations compared to inhaled soluble cisplatin, leading to much higher AUC values for liposomal cisplatin. Importantly, the higher AUC were also seen in bronchial lymph nodes for rats treated with liposomal cisplatin.

Lipids

The lipids used in the present invention can be synthetic, semi-synthetic or naturally-occurring lipids, including phospholipids, tocopherols, sterols, fatty acids, glycolipids, anionic lipids, cationic lipids. In terms of phosholipids, they can include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), sterically modified phosphatidylethanolamines, cholesterol derivatives, carotinoids, other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC, a major constituent of naturally-occurring lung surfactant. Other examples include dimyristoylphosphatidycholine (DMPC) and dimyristoylphosphatidylg-lycerol (DMPG) dipalmitoylphosphatidcholine (DPPC and dipalmitoylphosphatidylglycerol (DPPG) distearoylphosphatidylcholine (DSPC and distearoylphosphatidylglycerol (DSPG), dioleylphosphatidyl-ethanolamine (DOPE) and mixed phospholipids like palmitoylstearoylphosphatidylcholine (PSPC) and palmitoylstearolphosphatidylglycerol (PSPG), triacylglycerol, diacylglycerol, sphingosine, ceramide, sphingomyelin, single acylated phospholipids like mono-oleoyl-phosphatidylethanolamine (MOPE), and quadruple acylated.phospholipids like cardiolipin.

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate.

In a preferred embodiment of the invention the lipid composition contains 50 to 100 mol % DPPC and 0 to 50 mol % cholesterol. More preferably, the lipid complex contains 50 to 65 mol % DPPC and 35 to 50 mol % cholesterol.

Inhalation Devices

The inhalation delivery device of the inhalation system can be a nebulizer, a metered dose inhaler (MDI) or a dry powder inhaler (DPI). The device can contain and be used to deliver a single dose of the lipid compositions or the device can contain and be used to deliver multi-doses of the lipid compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically (including, for example, a vibrating porous membrane). The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer is employed to ensure the sizing of aqueous droplets containing the drug-lipid particles is optimal for positioning of the particle within, for example, the lungs. Typical droplet sizes for the nebulized lipid composition are from 1 to 5 microns.

For use with the nebulizer, the lipid composition preferably contains an aqueous component. Typically there is at least 80% by weight and preferably, at least 90% by weight of the aqueous component in the lipid composition to be administered with a nebulizer. The aqueous component may include for example, saline. In addition, the aqueous component may include up to 20% by weight of an aqueous compatible solvent such as ethanol.

Total administration time using a nebulizer will depend on the flow rate and the concentration of the cisplatin in the lipid composition. Variation of the total administration time is within the purview of those of ordinary skill in the art. Generally, the flow rate of the nebulizer will be at least 0.15 ml/min, for example, a flow rate of 0.2 ml/min is typical. By way of example, administration of a dose of 36 mg/m² of cisplatin using a lipid composition having a concentration of 1 mg/ml of cisplatin would be 4 hours (assuming a patient's body surface area is 2 m²). This administration time may, for example, be split into two administration sessions given over the course of one or two days to complete one treatment cycle. More preferably, administration of a dose of 36 mg/m² of cisplatin using a lipid composition having a concentration of 5 mg/ml of cisplatin would be 48 minutes to complete one treatment cycle.

In alternative embodiments, a metered dose inhaler (MDI) can be employed as the inhalation delivery device of the inhalation system. This device is pressurized (pMDI) and its basic structure consists of a metering valve, an actuator and a container. A propellant is used to discharge the formulation from the device. The composition can consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-1 1, 12 and 114 are used only when essential. The device of the inhalation system may deliver a single dose via, e.g., a blister pack, or it may be multi dose in design. The pressurized metered dose inhaler of the inhalation system can be breath actuated to deliver an accurate dose of the lipid based formulation. To insure accuracy of dosing, the delivery of the formulation may be programmed via a microprocessor to occur at a certain point in the inhalation cycle. The MDI may be portable and hand held.

In another alternative embodiment, a dry powder inhaler (DPI) can be used as the inhalation delivery device of the inhalation system. This device's basic design consists of a metering system, a powdered composition and a method to disperse the composition. Forces like rotation and vibration can be used to disperse the composition. The metering and dispersion systems may be mechanically or electrically driven and may be microprocessor programmable. The device may be portable and hand held. The inhaler may be multi or single dose in design and use such options as hard gelatin capsules, and blister packages for accurate unit doses. The composition can be dispersed from the device by passive inhalation; i.e., the patient's own inspiratory effort, or an active dispersion system may be employed. The dry powder of the composition can be sized via processes such as jet milling, spray dying and supercritical fluid manufacture. Acceptable excipients such as the sugars mannitol and maltose may be used in the preparation of the powdered formulations. These are particularly important in the preparation of freeze dried liposomes and lipid complexes. These sugars help in maintaining the liposome's physical characteristics during freeze drying and minimizing their aggregation when they are administered by inhalation. The hydroxyl groups of the sugar may help the vesicles maintain their tertiary hydrated state and help minimize particle aggregation.

The inventive method is particularly well-suited for the treatment of lung cancers, particularly, bronchoalveolar carcinoma, or carcinomatosis with lymphangitic spread. In addition, both primary and metastatic lung cancers are excellent candidates for the method of the invention.

Dosages

The dosage of any composition of the present invention will vary depending on the symptoms, age and body weight of the patient, the nature and severity of the disorder to be treated or prevented, the route of administration, and the form of the supplement. Any of the subject formulations may be administered in a single dose or in divided doses. Dosages for the compounds of the present invention may be readily determined by techniques known to those of skill in the art or as taught herein. Also, the present invention contemplates mixtures of more than one subject compound, as well as other therapeutic agents. Further, the present invention contemplates administration of the therapeutic agent that is contained in a subject coordination complex (or a related agent) in conjunction with the complex itself to increase the ratio of the therapeutic agent to the coordination complex formed upon release of the therapeutic agent,

In certain embodiments, the dosage of the subject compounds will generally be in the range of 0.01 ng to 10 g per kg body weight, specifically in the range of 1 ng to 0.1 g per kg, and more specifically in the range of 100 ng to 10 mg per kg.

An effective dose or amount, and any possible affects on the timing of administration of the formulation, may need to be identified for any particular compound of the present invention. This may be accomplished by routine experiment as described herein, using one or more groups of animals (preferably at least 5 animals per group), or in human trials if appropriate. The effectiveness of any compound and method of treatment or prevention may be assessed by administering the supplement and assessing the effect of the administration by measuring one or more indices associated with the neoplasm of interest, and comparing the post-treatment values of these indices to the values of the same indices prior to treatment.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compounds of the present invention, or alternatively other chemotherapeutic agents, may reduce the required dosage for any individual component because the onset and duration of effect of the different components may be complimentary. In such combined therapy, the different active agents may be delivered together or separately, and simultaneously or at different times within the day.

Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

Kits

This invention also provides kits for conveniently and effectively implementing the methods of this invention. Such kits comprise any of the compounds of the present invention or a combination thereof, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

The following examples further illustrate the present invention, but of course, should not be construed as in any way limiting its scope.

### EXEMPLIFICATION

### Example 1

Method of Preparing an Aqueous Cisplatin With Hither Potency Than its Aqueous Solubility Limit at Room Temperature

1) At temperatures 50-60° C., cisplatin in 0.9% sodium chloride solution at a level of 4 mg/ml and an ethanolic solution of 16 mg/ml DPPC and 8 mg/ml cholesterol at 55° C. are aseptically prepared.

2) The lipid solution is infused into the cisplatin solution while mixing the cisplatin solution.

3) After infusion, cisplatin/lipid dispersion is cooled down to 10° C. and then warmed up again to 50-60° C. for 15 min.

4) Step 3) is repeated 2-3 times.

5) The dispersion is aseptically washed with sterile 0.9% sodium chloride solution to remove residual ethanol and un-associated cisplatin via 500,000 MW cut-off membrane diafiltration unit.

After washing process, the dispersion provides 1 mg/ml cisplatin potency and concentrated to 3 mg/ml cisplatin and further concentrated to 5 mg/ml cisplatin by aseptically removing two third of the aqueous vehicle of 1 mg/ml product and four fifth of the aqueous vehicle of 1 mg/ml product, respectively. The removal of aqueous vehicle was carried out at a rate of 100 ml/min by diafiltration at 20° C. without compensating the permeate with fresh sterile 0.9% sodium chloride solution.

### Example 2

Inhalation for an extended time period is unfavorable to patients, especially with limited pulmonary function. High potency cisplatin dramatically reduces the treatment time, allowing each administration about an hour. For the dose of 36 mg/m², it allows patients to complete their treatment in one day instead of two days.

TABLE 3
Drug Concentration: 1.0 mg/ml cisplatin.

| | Number inhalation sessions | | | | |
|---|---|---|---|---|---|
| Dose | (example: body surface 2 m²) | Time | hrs. for | Total | No. |
| mg/m² | (fill volume 7 ml for 6 ml delivery) | (min.) | actual dose | duration* | days/cycle |
| 18.0 | 6 | 120 | 2 | 3.33 | 1 |
| 24.0 | 8 | 160 | 2.67 | 5.08 | 1 |
| 36.0 | 12 | 240 | 4 | 7.67 | 2 |

*total duration includes rest time between actual dose administration.

TABLE 4
Drug Concentration: 3 mg/ml cisplatin.

| | Number inhalation sessions | | | Total | |
|---|---|---|---|---|---|
| Dose | (example: body surface 2 m²) | Time | hrs. for | duration* | No. |
| mg/m² | (fill volume 7 ml for 6 ml delivery) | (min.) | actual dose | (min) | days/cycle |
| 18.0 | 2 | 40 | .67 | 45 | 1 |
| 24.0 | 2.67 | 53 | .89 | 65 | 1 |
| 36.0 | 4 | 80 | 1.33 | 150 | 1 |

*total duration includes rest time between actual dose administration(5 min between each nebulization and 1 hour after every three nebulizations).

### INCORPORATION BY REFERENCE

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of treating cancer in a patient comprising administering to the patient by inhalation a cancer treating effective amount of a lipid-based platinum compound formulation wherein the concentration of the platinum compound is greater than its aqueous solubility at temperatures no higher than 20° C.

2. The method of claim 1, wherein the platinum compound concentration is 1.2 mg/ml.

3. The method of claim 1, wherein the platinum compound concentration is 3 mg/ml.

4. The method of claim 1, wherein the platinum compound concentration is 5 mg/ml.

5. The method of claim 1, wherein the platinum compound is selected from the group consisting of: cisplatin, carboplatin(diammine(1,1-cyclobutanedicarboxylato)-platinum(II)), DACH-platinum, tetraplatin(ormaplatin)(tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

6. The method of claim 1, wherein the platinum compound is cisplatin.

7. The method of claim 1, wherein the lipid in the lipid-based platinum compound formulation is comprised of a member selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic aicd (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

8. The method of claim 1, wherein the lipid in the lipid-based platinum compound formulation is a mixture of a phospholipid and a sterol.

9. The method of claim 1, wherein the lipid in the lipid-based platinum compound formulation is a mixture of DPPC and cholesterol.

10. The method of claim 1, wherein the lipid in the lipid-based platinum compound formulation is a mixture of DPPC from 50 to 65 mol % and cholesterol from 35 to 50 mol %.

11. The method of claim 1, wherein the cancer is lung cancer.

12. The method of claim 11, wherein the lung cancer is Bronchoalveolar Carcinoma (BAC).

13. The method of claim 11, wherein the lung cancer is Lymphangitis carcinomatosis (LC).

14. The method of claim 11, wherein the lung cancer is Osteosarcoma metastatic to the lung.

15. The method of claim 1, wherein the ratio of platinum compound to lipid in the lipid-based platinum compound formulation is between 1:5 by weight and 1:50 by weight.

16. The method of claim 1, wherein the lipid-based platinum compound formulation comprises liposomes having a mean diameter of 0.01 microns to 3.0 microns.

17. The method of claim 1, wherein the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns.

18. The method of claim 1, wherein the lipid is a mixture of DPPC and cholesterol, the ratio of platinum compound to lipid in the lipid-based platinum compound formulation is between 1:5 by weight and 1:50 by weight, the lipid-based platinum compound formulation comprises liposomes having a mean diameter of 0.01 microns to 3.0 microns, and wherein the platinum compound is cisplatin.

19. The method of claim 1, wherein the lipid is a mixture of DPPC and cholesterol in a 2 to 1 ratio by weight, the ratio of platinum compound to lipid in the lipid-based platinum compound formulation is 1:20 by weight, the lipid-based platinum compound formulation comprises liposomes having a mean diameter of 0.40 microns, and wherein the platinum compound is cisplatin.

20. The method of claim 1, wherein the patient is a human.

21. The method of claim 1, wherein the lipid-based platinum compound formulation is administered to the patient at least once every three weeks.

22. The method of claim 1, wherein the lipid-based platinum compound formulation is administered to the patient at least once every two weeks.

23. The method of claim 1, wherein the amount of platinum compound in the lipid-based platinum compound formulation is 18 mg/m² or greater, 24 mg/m² or greater, 36 mg/m² or greater, or 48 mg/m² or greater.

24. The method of claim 1, wherein the amount of platinum compound in the lipid-based platinum compound formulation is 24 mg/m² or greater, and the lipid-based platinum compound formulation is administered to the patient at least once every three weeks.

25. The method of claim 1, wherein the amount of platinum compound in the lipid-based platinum compound formulation is 24 mg/m² or greater, and the lipid-based platinum compound formulation is administered to the patient at least once every two weeks.

26. A method of reducing the treatment time for the treatment of cancer in a patient comprising administering by inhalation to the patient a lipid-based platinum compound formulation wherein the concentration of the platinum compound is greater than its aqueous solubility at temperatures no higher than 20° C.

27. The method of claim 26, wherein the platinum compound concentration is 1.2 mg/ml.

28. The method of claim 26, wherein the platinum compound concentration is 3 mg/ml.

29. The method of claim 26, wherein the platinum compound concentration is 5 mg/ml.

30. The method of claim 26, wherein the platinum compound is selected from the group consisting of: cisplatin, carboplatin (diammine(1,1-cyclobutanedicarboxylato)-platinum(II)), DACH-platinum, tetraplatin(ormaplatin)(tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine) platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

31. The method of claim 26, wherein the platinum compound is cisplatin.

32. The method of claim 26, wherein the lipid in the lipid-based platinum compound formulation is comprised of a member selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic aicd (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic aicd (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

33. The method of claim 26, wherein the lipid in the lipid-based platinum compound formulation is a mixture of a phospholipid and a sterol.

34. The method of claim 26, wherein the lipid in the lipid-based platinum compound formulation is a mixture of DPPC and cholesterol.

35. The method of claim 26, wherein the lipid in the lipid-based platinum compound formulation is a mixture of DPPC from 50 to 65 mol % and cholesterol from 35 to 50 mol %.

36. The method of claim 26, wherein the cancer is lung cancer.

37. The method of claim 36, wherein the lung cancer is Bronchoalveolar Carcinoma (BAC).

38. The method of claim 36, wherein the lung cancer is Lymphangitis carcinomatosis (LC).

39. The method of claim 36, wherein the lung cancer is Osteosarcoma metastatic to the lung.

40. The method of claim 26, wherein the ratio of platinum compound to lipid in the lipid-based platinum compound formulation is between 1:5 by weight and 1:50 by weight.

41. The method of claim 26, wherein the lipid-based platinum compound formulation comprises liposomes having a mean diameter of 0.01 microns to 3.0 microns.

42. The method of claim 26, wherein the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns.

43. The method of claim 26, wherein the lipid is a mixture of DPPC and cholesterol, the ratio of platinum compound to lipid in the lipid-based platinum compound formulation is between 1:5 by weight and 1:50 by weight, the lipid-based platinum compound formulation comprises liposomes having a mean diameter of 0.01 microns to 3.0 microns, and wherein the platinum compound is cisplatin.

44. The method of claim 26, wherein the lipid is a mixture of DPPC and cholesterol in a 2 to 1 ratio by weight, the ratio of platinum compound to lipid in the lipid-based platinum compound formulation is 1:20 by weight, the lipid-based platinum compound formulation comprises liposomes having a mean diameter of 0.40 microns, and wherein the platinum compound is cisplatin.

45. The method of claim 26, wherein the patient is a human.

46. The method of claim 26, wherein the lipid-based platinum compound formulation is administered to the patient at least once every three weeks.

47. The method of claim 26, wherein the lipid-based platinum compound formulation is administered to the patient at least once every two weeks.

48. The method of claim 26, wherein the amount of platinum compound in the lipid-based platinum compound formulation is 18 mg/m² or greater, 24 mg/m² or greater, 36 mg/m² or greater, or 48 mg/m² or greater.

49. The method of claim 26, wherein the amount of platinum compound in the lipid-based platinum compound formulation is 24 mg/m² or greater, and the lipid-based platinum compound formulation is administered to the patient at least once every three weeks.

50. The method of claim 26, wherein the amount of platinum compound in the lipid-based platinum compound formulation is 24 mg/m² or greater, and the lipid-based platinum compound formulation is administered to the patient at least once every two weeks.
